(19) United States
(12) Patent Application Publication
(10) Pub. No.: US 2005/0266095 A1
(43) Pub. Date: Dec. 1, 2005

(54) Gentle preservative compositions

(76) Inventors: Erning Xia, Penfield, NY (US); Joseph C. Salamone, Fairport, NY (US)

Correspondence Address: Bausch & Lomb Incorporated, One Bausch & Lomb Place, Rochester, NY, 14604-2701, US

(21) Appl. No.: 10/858,116

(22) Filed: Jun. 1, 2004

(51) Int. Cl. A61K 33/40

(52) U.S. Cl. 424/616; 510/115

(57) ABSTRACT

Compositions are described which are useful in preserving any topically applied solution. Compositions including urea-hydrogen peroxide complex in solution in an amount effective to preserve contact lenses are also described, as well as methods of making and using such solutions. A hydrogen peroxide component of such solutions, when introduced into the eye, is converted to oxygen and water, leaving only the natural product, urea.

### FIELD OF THE INVENTION

The present invention is directed toward the use of urea-hydrogen peroxide complex (UHPC) in the manufacture of gentle preservative systems. More particularly, the present invention is directed toward the use of compositions including hydrogen peroxide molecules trapped in cages of urea molecules via hydrogen bonding to provide gentle preservation of ophthalmic solutions and medical devices.

### BACKGROUND OF THE INVENTION

Contact lenses in wide use today fall into two general categories, hard and soft. The hard or rigid corneal type lenses are formed from materials prepared by the polymerization of acrylic esters, such as poly(methyl methacrylate) (PMMA). The gel, hydrogel or soft type lenses are made by polymerizing such monomers as 2-hydroxyethyl methacrylate (HEMA) or, in the case of extended wear lenses, by polymerizing silicon-containing monomers or macromonomers. Both the hard and soft types of contact lenses are exposed to a broad spectrum of microbes during normal wear and become soiled relatively quickly. Contact lenses whether hard or soft therefore require routine cleaning and disinfecting. Failure to routinely clean and disinfect contact lenses properly can lead to a variety of problems ranging from mere discomfort when being worn to serious ocular infections. Ocular infections caused by virulent microbes such as Pseudomonas aeruginosa can lead to loss of the infected eye(s) if left untreated or if allowed to reach an advanced stage before initiating treatment.

U.S. Pat. No. 4,758,595 discloses a contact lens disinfectant and preservative containing a biguanide or a water-soluble salt thereof in combination with a buffer, preferably a borate buffer, e.g., boric acid, sodium borate, potassium tetraborate, potassium metaborate or mixtures of the same.

U.S. Pat. No. 4,361,548 discloses a contact lens disinfectant and preservative containing dilute aqueous solutions of a polymer; namely, dimethyldiallylammonium chloride (DMDAAC) having molecular weights ranging from about 10,000 to 1,000,000. Amounts of DMDAAC homopolymer as low as 0.00001 percent by weight may be employed when an enhancer, such as sorbic acid or a phenylmercuric salt is used therewith. Although lens binding and concomitant eye tissue irritation with DMDAAC were reduced, it was found in some users to be above desirable clinical levels.

Despite the availability of various commercially available contact lens disinfecting systems such as heat, hydrogen peroxide, organoperoxide derivatives, inorganic peroxide derivatives, biguanides, polymeric biguanides, quaternary ammonium salts of low and high molecular weight, amidoamines and other chemical agents, there continues to be a need for improved disinfecting and/or preserving systems. Such improved disinfecting and/or preserving systems include systems that are simple to use, are effective against a broad spectrum of microbes, are non-toxic and do not cause ocular irritation as the result of binding to the contact lens material. There is a particular need in the field of contact lens disinfection and ophthalmic composition preservation for safe and effective chemical agents with antimicrobial activity.

### SUMMARY OF THE INVENTION

The present invention relates to unique, gentle, self-preserving solutions such as for example but not limited to ophthalmic solutions and like solutions useful for topical application. Such self-preserving solutions may be useful for cleaning, soaking, rinsing, wetting and conditioning all types of contact lenses, including rigid permeable contact lenses, for nasal sprays, for ear drops, for eye drops and the like. It has been found that solutions containing urea-hydrogen peroxide complex (UHPC), available commercially from Sigma Aldrich Corporation, St. Louis, Mo., exhibit excellent preservative effect, while also, in the case of contact lens solution use, increasing lens wearer comfort. The UHPC-containing compositions of the present invention are also useful for the preservation of ophthalmic solutions, pharmaceuticals, artificial tears, comfort drops and the like against microbial contamination.

The subject UHPC-containing compositions are effective preservatives useful in the manufacture of topical solutions that are non-toxic, simple to use and do not cause ocular irritation. UHPC-containing compositions of the present invention provide a preservative system wherein the hydrogen peroxide component is converted to oxygen and water in the eye, leaving the natural product, urea.

Accordingly, it is an object of the present invention to provide compositions with enhanced biocidal activity useful in the manufacture of self-preserving ophthalmic systems.

Another object of the present invention is to provide a method for using compositions with enhanced biocidal activity in the preservation of medical devices.

Another object of the present invention is to provide compositions with enhanced biocidal activity useful in ophthalmic systems to preserve contact lenses.

Another object of the present invention is to provide compositions with enhanced biocidal activity useful in preserving ophthalmic systems from microbial contamination.

Another object of the present invention is to provide compositions with enhanced biocidal activity useful in ophthalmic systems for preserving contact lenses with reduced or eliminated eye irritation.

Another object of the present invention is to provide a method of making gentle compositions having biocidal activity useful in preserving ophthalmic systems.

Still another object of the present invention is to provide a method of using gentle compositions with biocidal activity as preservative agents.

These and other objectives and advantages of the present invention, some of which are specifically described and others that are not, will become apparent from the detailed description and claims that follow.

### DETAILED DESCRIPTION OF THE INVENTION

Compositions of the present invention can be used with all contact lenses such as conventional hard and soft lenses, as well as rigid and soft gas permeable lenses. Such suitable lenses for use with compositions of the present invention include both hydrogel and non-hydrogel lenses, as well as silicone and fluorine-containing lenses. The term “soft contact lens” as used herein generally refers to those contact lenses that readily flex under small amounts of force. Typically, soft contact lenses are formulated from polymers having a certain proportion of repeat units derived from monomers such as 2-hydroxyethyl methacrylate and/or other hydrophilic monomers, typically crosslinked with a crosslinking agent. However, newer soft lenses, especially for extended wear, are being made from high-Dk silicone-containing materials.

Compositions of the present invention comprise urea-hydrogen peroxide complex (UHPC), which is a white solid, very soluble in water. The UHPC-containing compositions of the present invention are useful in the production of self-preserving solutions. The self-preserving solutions are useful in preserving medical devices, pharmaceuticals, topically applied solutions and the like from microbial contamination. For example, the subject UHPC-containing compositions are useful in preserving contact lens care solutions employed in cleaning, soaking, rinsing and/or wetting contact lenses. Compositions of the present invention are preferably in solution in sufficient concentration to destroy harmful microorganisms and thus preserve the solution from microbial contamination throughout the intended shelf-life of the solution.

Compositions of the present invention in solution are physiologically compatible or “ophthalmically safe” for use with contact lenses. An ophthalmically safe solution has a tonicity and pH that is compatible with the eye and comprises materials, and amounts thereof, that are non-cytotoxic according to ISO (International Standards Organization) standards and U.S. FDA (Food and Drug Administration) regulations. Ophthalmically safe as used herein means that a contact lens treated with or in the subject solution is generally suitable and safe for direct placement on the eye without rinsing. The subject solutions are safe and comfortable for daily contact with the eye via a contact lens that has been wetted with the solution. Solutions of the present invention are particularly safe and comfortable since the hydrogen peroxide component thereof is converted to oxygen and water in the eye, leaving only the natural product, urea. Solutions of the present invention are sterile in that the absence of microbial contaminants in the product prior to release should be statistically demonstrated to the degree necessary for such products.

As noted above, compositions of the present invention include hydrogen peroxide molecules trapped in cages of urea molecules via hydrogen bonding to form an urea-hydrogen peroxide complex (UHPC). The UHPC is present in the subject compositions in a total amount sufficient to achieve preservative efficacy, i.e., a “preservative amount”, preferably from approximately 0.0001 to approximately 15.0 percent by weight based on the total weight of the composition, but more preferably from about 0.001 to about 10.0 percent by weight.

In addition to UHPC, compositions of the present invention may optionally include one or more aminoalcohol buffers, such as for example but not limited to ethanolamine buffers present in a total amount of from approximately 0.02 to approximately 3.0 percent by weight based on the total weight of the composition. Suitable aminoalcohol buffers include for example but are not limited to monoethanolamine (MEA), diethanolamine (DEA), triethanolamine (TEA), 2-amino-2-methyl-1,3-propanediol (AMPD), 2-dimethylamino-2-methyl-1-propanediol (DMAMP), 2-amino-2-ethylpropanol (AEP), 2-amino-1-butanol (AB) and 2-amino-2-methyl-1-propanol (AMP), but preferably MEA, DEA or TEA.

Compositions of the present invention may likewise include one or more buffers, or buffering systems, in addition to the aminoalcohol buffers described above, if any, to adjust the final pH of the solution. Suitable buffers or buffering systems include for example but are not limited to phosphate buffers, borate buffers, citrate buffers, tris(hydroxymethyl)aminomethane (Tris) buffers, bis(2-hydroxyethyl)-imino-tris(hydroxymethyl)methane (bis-Tris) buffers, sodium bicarbonate, and combinations thereof. A suitable buffering system for example may include at least one phosphate buffer and at least one borate buffer, which buffering system has a buffering capacity of 0.01 to 0.5 mM, preferably 0.03 to 0.45, of 0.01 N of HCl and 0.01 to 0.3, preferably 0.025 to 0.25, of 0.01 N of NaOH to change the pH one unit. Buffering capacity is measured by a solution of the buffers only. The pH of lens care solutions of the present invention is preferably maintained within the range of 6.0 to 9.0, more preferably about 6.5 to 9.0, most preferably about 7.4 to 8.0.

Compositions of the present invention may likewise optionally include one or more tonicity agents to approximate the osmotic pressure of normal lachrymal fluids, which is equivalent to a 0.9 percent solution of sodium chloride or 2.5 percent glycerin solution. Examples of suitable tonicity agents include but are not limited to sodium and potassium chloride, dextrose, mannose, glycerin, calcium and magnesium chloride. These agents are typically used individually in amounts ranging from about 0.01 to 2.5 percent weight per volume and preferably, from about 0.2 to about 1.5 percent weight per volume. Preferably, the tonicity agent is employed in an amount to provide a final osmotic value of 200 to 450 mOsm/kg and more preferably between about 220 to about 350 mOsm/kg, and most preferably between about 220 to about 320 mOsm/kg.

Compositions of the present invention may also include one or more chelating agents to bind traces of reducing metal impurities. Suitable chelating agents include for example but are not limited to ethylenediaminetetraacetic acid (EDTA), salts of EDTA and phosphonates commercially available under the trade name Dequest™ (Monsanto, St. Louis, Mo.). Chelating agents are preferably used in amounts ranging from about 0.01 to about 0.2 weight percent.

Compositions of the present invention may likewise include one or more additive agents. Suitable additive agents include for example but are not limited to polyols such as glycerin, polysaccharide(s) and polyether(s), which may be added based on particular wetting, conditioning and/or cleaning properties. Additive agents may be present in the subject compositions in a total amount of from approximately 0.001 to approximately 5.0 percent by weight based on the total weight of the composition, but more preferably from about 0.1 to about 0.5 percent by weight.

Suitable polyethers include for example but are not limited to polyethers based upon poly(ethylene oxide)-poly(propylene oxide)-poly(ethylene oxide), i.e., (PEO-PPO-PEO), or poly(propylene oxide)-poly(ethylene oxide)-poly(propylene oxide), i.e., (PPO-PEO-PPO), or a combination thereof. PEO-PPO-PEO and PPO-PEO-PPO are commercially available under the trade names Pluronics™, R-Pluronics™, Tetronics™ and R-Tetronics™ (BASF Wyandotte Corp., Wyandotte, Mich.) and are further described in U.S. Pat. No. 4,820,352 incorporated herein in its entirety by reference. Suitable polyethers for use in the present composition should be soluble in solution, should not become turbid, and should be non-irritating to eye tissues.

Suitable polysaccharides include for example but are not limited to cellulose polymers like hydroxyethyl cellulose, hydroxypropyl cellulose, carboxymethyl cellulose, povidone, poly(vinyl alcohol), variations of polyquaternium-10 such as for example Polymer JR™ (Dow Chemical Company, Midland Mich.) and the like.

The compositions of the present invention are described in still greater detail in the examples that follow.

### EXAMPLE 1

### Preservative System Preparation

Sample solutions were prepared in accordance with the present invention for testing. Formulations of the sample solutions are set forth below in Table 1.

TABLE 1
Sample Solutions

| Ingredients | Solutions | | |
|---|---|---|---|
| W/W Percent | 1 | 2 | 3 |
| Triethanolamine HCL | 0.937 | 0.937 | 0.937 |
| Triethanolamine BASE | 0.149 | 0.149 | 0.149 |
| Pluronic F127 | 2.000 | 2.000 | 2.000 |
| Tetronic 1107 | 1.000 | 1.000 | 1.000 |
| Glycerin | 1.000 | 1.000 | 1.000 |
| Sodium Chloride | 0.030 | 0.030 | 0.030 |
| EDTA | 0.025 | 0.025 | 0.050 |
| UHPC | 0.050 | 0.100 | 0.050 |
| Polymer JR | 0.020 | 0.020 | 0 |
| pH | 7.0-7.4 | 7.0-7.4 | 7.0-7.4 |
| Osmolality (mOsm/Kg) | 240-280 | 240-280 | 240-280 |
| Ingredients | Solutions | | |
| W/W Percent | 4 | 5 | 6 |
| Triethanolamine HCL | 0.937 | 0.937 | 0.937 |
| Triethanolamine BASE | 0.149 | 0.149 | 0.149 |
| Pluronic F127 | 2.000 | 2.000 | 2.000 |
| Tetronic 1107 | 1.000 | 1.000 | 1.000 |
| Glycerin | 1.000 | 1.000 | 1.000 |
| Sodium Chloride | 0.030 | 0.030 | 0.030 |
| EDTA | 0.050 | 0.025 | 0.025 |
| UHPC | 0.100 | 0.050 | 0.100 |
| Polymer JR | 0 | 0.010 | 0.010 |
| pH | 7.0-7.4 | 7.0-7.4 | 7.0-7.4 |
| Osmolality (mOsm/Kg) | 240-280 | 240-280 | 240-280 |

### EXAMPLE 2

### ISO/FDA Microbial Preservative Efficacy Testing of Sample Solutions with Five of FDA/ISO Challenge Microorganisms

Sample solutions prepared in accordance with Example 1 above, were each tested for ISO/FDA microbial preservative efficacy using five FDA/ISO challenge microorganisms, i.e., three bacteria and two fungi. Acceptance criteria established for bacteria require that the number of viable bacteria, recovered per ml, is reduced by not less than 3.0 logs at 14 days. After the rechallenge at day 14, the concentration of bacteria shall be reduced by at least 3.0 logs by day 28. Acceptance criteria established for yeasts and molds require that the number of viable yeasts and molds, recovered per ml, remain at or below initial concentrations within an experimental error of ±0.5 logs within 14 days. After day 28, the concentration of mold and yeast shall remain at or below the concentrations after rechallenge within an experimental error of ±0.5 logs. Results of the ISO/FDA microbial preservative efficacy testing of the subject sample solutions are set forth below in Table 2. The results set forth in Table 2 illustrate that in the presence of UHPC, an antimicrobial agent is not required to achieve effective solution preservation.

TABLE 2
Results of ISO/FDA Microbial Preservative Efficacy Testing
Preservative Efficacy

| | | Log Reduction of | | |
|---|---|---|---|---|
| | | Solution | | |
| ISO Agent | Days | 1 | 2 | 3 |
| Staphylococcus aureus | 7 | >4.9 | >4.9 | >4.9 |
| (ATCC 6538) | 14 | >4.9 | >4.9 | >4.9 |
| | 21 | >3.9 | >3.9 | >3.9 |
| | 28 | 3.9 | 3.8 | >3.9 |
| Pseudomonas aeruginosa | 7 | >4.9 | >4.9 | >4.9 |
| (ATCC 9027) | 14 | >4.9 | >4.9 | >4.9 |
| | 21 | >4.0 | >4.0 | >4.0 |
| | 28 | >4.0 | >4.0 | >4.0 |
| Escherichia coli | 7 | >4.9 | >4.9 | >4.9 |
| (ATCC 8739) | 14 | >4.9 | >4.9 | >4.9 |
| | 21 | >3.8 | >3.8 | >3.8 |
| | 28 | >3.8 | >3.8 | >3.8 |
| Candida albicans | 7 | >4.9 | >4.9 | 3.1 |
| (ATCC 10231) | 14 | >4.9 | 4.9 | 4.7 |
| | 21 | 1.1 | 3.6 | 0.8 |
| | 28 | 2.1 | 3.9 | 1.6 |
| Aspergillus niger | 7 | 0.0 | 0.1 | 0.0 |
| (ATCC 16404) | 14 | 0.0 | −0.1 | 0.0 |
| | 21 | 0.0 | 0.1 | 0.0 |
| | 28 | 0.0 | 0.1 | 0.1 |
| | | Log Reduction of | | |
| | | Solution | | |
| ISO Agent | Days | 4 | 5 | 6 |
| Staphylococcus aureus | 7 | >4.9 | >4.9 | >4.9 |
| (ATCC 6538) | 14 | >4.9 | >4.9 | >4.9 |
| | 21 | >3.9 | >3.9 | >3.9 |
| | 28 | 3.9 | >3.9 | >3.9 |
| Pseudomonas aeruginosa | 7 | >4.9 | >4.9 | >4.9 |
| (ATCC 9027) | 14 | >4.9 | >4.9 | 4.9 |
| | 21 | >4.0 | >4.0 | >4.0 |
| | 28 | >4.0 | 3.9 | >4.0 |
| Escherichia coli | 7 | 4.5 | 4.9 | >4.9 |
| (ATCC 8739) | 14 | >4.9 | >4.9 | >4.9 |
| | 21 | >3.8 | >3.8 | >3.8 |
| | 28 | >3.8 | >3.8 | >3.8 |
| Candida albicans | 7 | >4.9 | 3.5 | >4.9 |
| (ATCC 10231) | 14 | >4.9 | >4.9 | >4.9 |
| | 21 | 2.8 | 1.1 | 3.3 |
| | 28 | 3.7 | 2.4 | 3.8 |
| Aspergillus niger | 7 | 0.0 | 0.1 | 0.0 |
| (ATCC 16404) | 14 | 0.0 | −0.1 | 0.0 |
| | 21 | 0.1 | 0.0 | 0.0 |
| | 28 | 0.1 | 0.1 | 0.1 |

UHPC-containing compositions of the present invention are useful in contact lens care solutions for preserving contact lenses. A preserving amount of UHPC is an amount that will at least partially reduce the microorganism population in the formulations employed. Preferably, a preserving amount is that which will reduce the microbial burden of representative bacteria by two log orders in four hours and more preferably by one log order in one hour. Most preferably, a preserving amount is an amount that will eliminate the microbial burden on a contact lens when used according to its regimen for the recommended soaking time ISO (International Standards for Ophthalmic Optics)/FDA Stand-Alone Procedures for Disinfection Test (ISO/DIS 14729; 2001). Unexpectedly, in the presence of UHPC, a antimicrobial agent is not required to achieve effective solution preservation.

As stated above, contact lenses are preserved by contacting the lenses with a solution containing an effective amount of one or more compositions of the present invention. Although this may be accomplished by simply soaking lenses in a solution containing one or more compositions of the present invention, enhanced efficacy can be achieved if a few drops of a solution of the present invention are initially placed on each side of the lens, and rubbing the lens for a period of time, for example, approximately 20 seconds. The lens can then be subsequently immersed within several milliliters of the subject solution. Preferably, the lens is permitted to soak in the solution for at least four hours. The lenses are then removed from the solution, rinsed with the same or a different solution, for example a preserved isotonic saline solution made in accordance with the present invention, and then replaced on the eye.

Solutions containing one or more compositions of the present invention may be formulated into specific contact lens care products for use as customary in the field of ophthalmology. Such products include but are not limited to wetting solutions, soaking solutions, cleaning and conditioning solutions, as well as in-eye cleaning and conditioning solutions.

While the invention has been described in conjunction with specific examples thereof, this is illustrative only. Accordingly, many alternatives, modifications, and variations will be apparent to those skilled in the art in the light of the foregoing description and it is, therefore, intended to embrace all such alternatives, modifications, and variations as to fall within the spirit and scope of the appended claims.

1. A composition comprising:
urea-hydrogen peroxide complex in an amount effective for solution preservation.

2. A composition comprising:
urea-hydrogen peroxide complex in an amount effective as a preserving agent.

3. The composition of claim 1 or 2 wherein said urea-hydrogen peroxide complex is present in about 0.0001 to about 15.0 percent by weight.

4. The composition of claim 1 or 2 wherein said urea-hydrogen peroxide complex is present in about 0.001 to about 10.0 percent by weight.

5. The composition of claim 1 or 2 additionally comprising one or more polysaccharides selected from the group consisting of cellulose polymers, povidone, poly(vinyl alcohol) and variations of polyquaternium-10.

6. The composition of claim 1 or 2 additionally comprising a variation of polyquaternium-10.

7. The composition of claim 1 or 2 additionally comprising one or more chelating agents.

8. The composition of claim 1 or 2 additionally comprising one or more chelating agents selected from the group consisting of ethylenediaminetetraacetic acid, salts of ethylenediaminetetraacetic acid and phosphonates.

9. The composition of claim 1 or 2 additionally comprising the chelating agent ethylenediaminetetraacetic acid.

10. The composition of claim 1 or 2 additionally comprising one or more tonicity agents.

11. The composition of claim 1 or 2 additionally comprising one or more additive agents.

12. The composition of claim 1 or 2 additionally comprising one or more buffers or buffering systems.

13. The composition of claim 1 or 2 additionally comprising one or more aminoalcohol buffers.

14. The composition of claim 1 or 2 wherein said composition has a pH of about 6.0 to 9.0

15. The composition of claim 1 or 2 wherein said composition has an osmolality of about 200 to 450 mOsmo/Kg.

16. A method of producing the composition of claim 1 or 2 comprising:
providing urea-hydrogen peroxide complex in said composition in an amount effective for preservation.

17. The method of claim 16 wherein said method additionally comprises providing one or more polysaccharides in said composition.

18. The method of claim 16 wherein said method additionally comprises providing polyquaternium-10 in said composition.

19. The method of claim 16 wherein said method additionally comprises providing one or more aminoalcohol buffers in said composition.

20. The method of claim 16 wherein said method additionally comprises providing one or more buffers or buffering systems in said composition.

21. The method of claim 16 wherein said method additionally comprises providing one or more additive agents in said composition.

22. The method of claim 16 wherein said method additionally comprises providing one or more chelating agents in said composition.

23. A solution comprising one or more compositions of claim 1 or 2.

24. The solution of claim 23 wherein said solution includes one or more buffers or buffering systems.

25. The solution of claim 23 wherein said solution includes one or more additive agents.

26. The solution of claim 23 wherein said solution includes one or more chelating agents.

27. The solution of claim 23 wherein said solution includes one or more aminoalcohol buffers.

28. A method of using the solution of claim 23 comprising:
contacting a surface of a contact lens with said solution for a period of time suitable to eliminate a microbial burden on said contact lens.

29. A method of using the solution of claim 23 comprising:
contacting a surface of a medical device with said solution for a period of time suitable to eliminate a microbial burden on said medical device.

30. A method of producing the solution of claim 23 comprising:
adding an effective amount of urea-hydrogen peroxide complex to a solution.

31. A composition for preserving a contact lens comprising:
an effective amount of urea-hydrogen peroxide complex.

32. The composition of claim 31 wherein said urea-hydrogen peroxide complex is present in about 0.0001 to about 15.0 percent by weight.

33. The composition of claim 31 wherein said urea-hydrogen peroxide complex is present in about 0.001 to about 10.0 percent by weight.

34. The composition of claim 31 additionally comprising one or more polysaccharides selected from the group consisting of cellulose polymers, povidone, poly(vinyl alcohol) and variations of polyquaternium-10.

35. The composition of claim 31 additionally comprising a variation of polyquaternium-10.

36. The composition of claim 31 additionally comprising one or more chelating agents.

37. The composition of claim 31 additionally comprising one or more chelating agents selected from the group consisting of ethylenediaminetetraacetic acid, salts of ethylenediaminetetraacetic acid and phosphonates.

38. The composition of claim 31 additionally comprising the chelating agent ethylenediaminetetraacetic acid.

39. The composition of claim 31 additionally comprising one or more tonicity agents.

40. The composition of claim 31 additionally comprising one or more additive agents.

41. The composition of claim 31 additionally comprising one or more buffers or buffering systems.

42. The composition of claim 31 additionally comprising one or more aminoalcohol buffers.

43. The composition of claim 31 wherein said composition has a pH of about 6.0 to 9.0

44. The composition of claim 31 wherein said composition has an osmolality of about 200 to 450 mOsmo/Kg.

45. A solution for preserving a contact lens comprising:
an effective amount of urea-hydrogen peroxide complex in solution.

46. The solution of claim 45 wherein said urea-hydrogen peroxide complex is present in about 0.0001 to about 15.0 percent by weight.

47. The solution of claim 45 wherein said urea-hydrogen peroxide complex is present in about 0.001 to about 10.0 percent by weight.

48. The solution of claim 45 additionally comprising one or more polysaccharides selected from the group consisting of cellulose polymers, povidone, poly(vinyl alcohol) and variations of polyquaternium-10.

49. The solution of claim 45 additionally comprising a variation of polyquaternium-10.

50. The solution of claim 45 additionally comprising one or more chelating agents.

51. The solution of claim 45 additionally comprising one or more chelating agents selected from the group consisting of ethylenediaminetetraacetic acid, salts of ethylenediaminetetraacetic acid and phosphonates.

52. The solution of claim 45 additionally comprising the chelating agent ethylenediaminetetraacetic acid.

53. The solution of claim 45 additionally comprising one or more tonicity agents.

54. The solution of claim 45 additionally comprising one or more additive agents.

55. The solution of claim 45 additionally comprising one or more buffers or buffering systems.

56. The solution of claim 45 additionally comprising one or more aminoalcohol buffers.

57. The solution of claim 46 wherein said composition has a pH of about 6.0 to 9.0

58. The solution of claim 46 wherein said composition has an osmolality of about 200 to 450 mOsmo/Kg.
